(19) United States
(12) Patent Application Publication
(10) Pub. No.: US 2016/0151022 A1
(43) Pub. Date: Jun. 2, 2016

(54) AUTOMATED IDENTIFICATION OF PHYSIOLOGICAL DATA

(71) Applicant: COVIDIEN LP, Mansfield, MA (US)

(72) Inventors: David B. Berlin, Niwot, CO (US); Andy S. Lin, Boulder, CO (US)

(21) Appl. No.: 14/954,538

(22) Filed: Nov. 30, 2015

Related U.S. Application Data
(60) Provisional application No. 62/085,985, filed on Dec. 1, 2014.

(51) Int. Cl. A61B 5/00; A61B 5/024; A61B 5/1455; A61B 5/0402

(52) U.S. Cl. CPC A61B 5/7246; A61B 5/0402; A61B 5/02416; A61B 5/14551; A61B 5/0006

(57) ABSTRACT

A method for associating physiological sensors to patients includes acquiring associated physiological data from a first physiological sensor associated with an identified patient. The method also includes acquiring unassociated physiological data from a second physiological sensor associated with an unidentified patient. The method further includes comparing a parameter in the unassociated physiological data to a common or correlated parameter in the associated physiological data to determine if the second physiological sensor is associated with the identified patient.

### CROSS-REFERENCE TO RELATED APPLICATIONS

The present application claims the benefit of U.S. Provisional Application No. 62/085,985, which was filed on Dec. 1, 2014, and entitled “AUTOMATED IDENTIFICATION OF PHYSIOLOGICAL DATA”, the disclosure of which is incorporated herein by reference in its entirety.

### BACKGROUND

The present disclosure relates generally to techniques for associating physiological sensors with patients and, more particularly, to automated identification of physiological data and association of the physiological data to one or more patients.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Medical monitors are used to capture data about a patient's physiology to allow caregivers to monitor the patient's clinical condition. The medical monitor may include or be coupled to one or more sensors attached to the body of the patient that detect and monitor physiological parameters of the patient. A variety of types of medical monitors and sensors may be used to detect and display the patient's vital signs and/or other physiological parameters. For example, a pulse oximeter sensor may be attached to the patient (e.g., a finger) to detect and monitor the patient's functional oxygen saturation of arterial hemoglobin (i.e., SpO₂) and heart rate. An electrocardiography (ECG) sensor may be attached to the patient (e.g., the chest) to detect and monitor the patient's heart rate, respiratory rate, and electrocardiogram signals.

Physiological data generated by the medical monitor or the sensor may be associated with a particular patient. For example, the association may include a manual association, e.g., a caregiver may manually input a patient's identification information (e.g., name, admission number, bed number, or the like) to a medical monitor when attaching the sensor to the patient, thereby associating the patient's identification information with the collected physiological data from the medical monitor or the corresponding sensor. In turn, the identification information may be provided along with the sensor data to a central monitoring station that displays information related to a number of patients. In addition, this associated data may be collected and stored as part of the patient's medical record. However, an individual monitor or sensor may initially be used for one patient but may also be reused and associated with a different patient at a later time. As such, it may be time consuming and inefficient to manually associate a patient's identification information to the medical monitor (or the sensor, or the physiological data collected thereby) each time a medical monitor or sensor is reattached to the same patient or a reused for a different patient.

Further, identification of a wireless sensor with a particular monitor may be challenging. Many sensors and monitors communicate wirelessly with one another, and a medical monitor may receive a number of incoming signals from patients in surrounding beds that are within a signal transmission area. Accordingly, determining if an incoming wireless signal is from a desired patient may be complex in the context of several incoming signals that are associated with a particular sensor type.

### BRIEF DESCRIPTION OF THE DRAWINGS

Advantages of the disclosed techniques may become apparent upon reading the following detailed description and upon reference to the drawings in which:

FIG. 1 is a schematic diagram of a technique for associating a physiological sensor to a patient in accordance with embodiments of the present disclosure;

FIG. 2 is a schematic diagram of physiological data from two physiological sensors used for the technique of FIG. 1;

FIG. 3 is a schematic diagram of a technique for comparing the physiological data from two physiological sensors in accordance with embodiments of the present disclosure;

FIG. 4 is a schematic diagram of a technique for comparing the physiological data from two physiological sensors in accordance with embodiments of the present disclosure;

FIG. 5 is a schematic diagram of a technique for synchronizing and comparing the physiological data from two physiological sensors in accordance with embodiments of the present disclosure;

FIG. 6 is a block diagram of a medical monitoring system in accordance with embodiments of the present disclosure;

FIG. 7 is a block diagram of a central monitoring station in accordance with embodiments of the present disclosure;

FIG. 8 is a flow diagram of a method for associating a physiological sensor to a patient in accordance with embodiments of the present disclosure;

FIG. 9 is a flow diagram of a method for associating a physiological sensor to a patient in accordance with embodiments of the present disclosure; and

FIG. 10 is a flow diagram of a method for associating a physiological sensor to a patient in accordance with embodiments of the present disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In a patient care setting, sensors attached to a particular patient generate physiological parameter data that is used by a caregiver to assess the patient's condition. However, as patient monitoring becomes more complex, a given patient may be monitored by several different sensors, and each of these sensors may be coupled to one or more monitors. Accordingly, collecting all of the data associated with a given patient may involve gathering data from different sensor and/or monitor types. Further, depending on the capabilities of each monitor, each sensor may be associated with its patient using a variety of different methods, including handwritten tags on the sensor, manual inputs to the monitor, patient data stored on a memory of the sensor, barcodes, etc. In addition, some sensors may not have any method associating them with a patient other than their physical proximity to the patient.

Provided herein are techniques for automatically associating a physiological sensor with a patient in a system with multiple physiological sensors. The disclosed techniques improve caregiver efficiency by reducing or eliminating manual input time and notetaking to collect or associate sensor data with a patient. In the disclosed embodiments, the patient's own physiological parameters may be used as an identification key for determining which, if any, additional sensors are associated with a particular patient. For example, a patient's heart rate may be determined by multiple sensors types, including pulse oximetry sensors and EKG (ECG) sensors. Accordingly, the heart rate is an overlapping parameter or a common feature between these two sensor types and, if these sensors are applied to the same patient, the measured heart rate over the same time period should be the approximately the same, plus or minus a measurement tolerance of each sensor type. In one embodiment, if a patient already is being monitored via pulse oximetry, and the measured heart rate is, for example, 60 per minute, over a given time window, then any EKG sensors applied to the patient should output a similar heart rate of 60 per minute over the same time window. Accordingly, the common feature of the heart rate is matched for these sensors applied to the same patient. In another embodiment, a variability or sequence of the overlapping or common feature between different sensor types may be used in addition to or instead of the overlapping or common feature itself. For example, if a pulse oximetry sensor detects that a patient has variable heart rates during a period of time with a number of one-second time intervals (e.g., 60, 61, 62, 60, . . . per minute in the first, second, third, fourth, . . . second, respectively), then any EKG sensors applied to the patient should output a similar or same (plus or minus a measurement tolerance) variability of the detected heart rate during the same period of time.

The disclosed techniques use overlapping or common features and/or a sequence or variability thereof to associate sensor signals from different sensors with a particular patient. For example, if an unassociated EKG signal outputs a heart rate that is close to or matching a heart rate for a sensor already associated with the patient, a monitoring system may flag the unassociated EKG signal as being a likely match for that patient and may associate the EKG signal with the patient identification data already in place for the pulse oximetry data. As such, the techniques disclosed herein may provide an automated and streamlined approach for clinical care providers to associate data from physiological sensors with one or more patients, or patients' records. Repetitive and manual associations of one patient with multiple applied physiological sensors may be reduced or eliminated. In addition, the techniques disclosed herein may provide a safety check to validate that data from disparate physiological sensors is tied back to the appropriate patient record. Inefficiency and manual error in association of physiological data to patients' records may be reduced or eliminated.

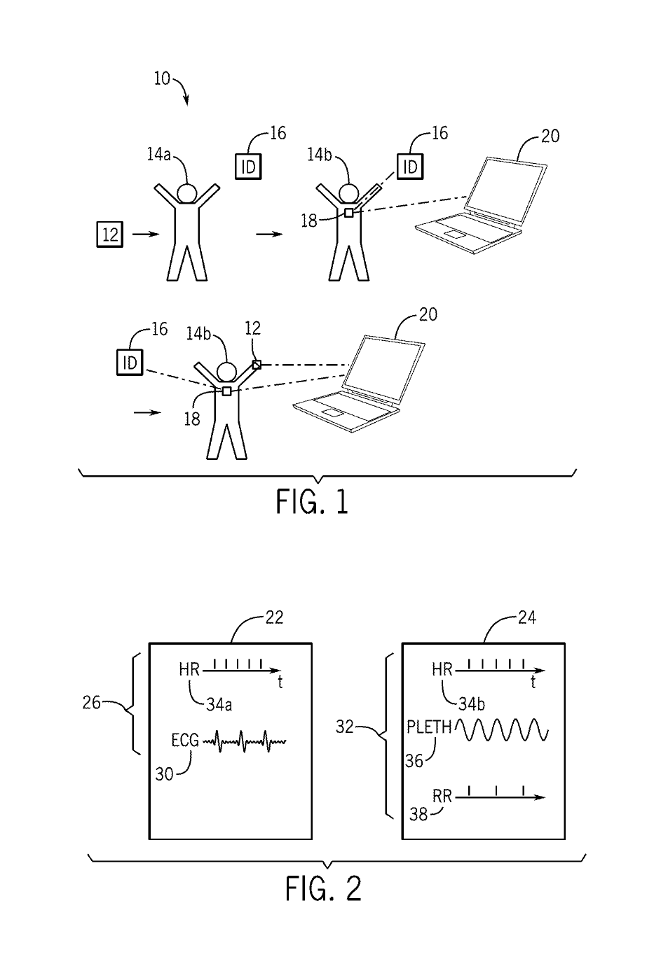

With the foregoing in mind, FIG. 1 illustrates a schematic diagram of a technique 10 for associating an unassociated physiological sensor 12 with a patient 14 and the patient's corresponding identification data 16 in accordance with the present disclosure. In certain embodiments, an associated or identified physiological sensor 18 is coupled to an identified patient 14b to detect and monitor one or more physiological parameters of the patient 14b. The identification data 16 (e.g., name, gender, date of birth, admission date, provider information, room number, or the like) pertinent to the patient may be associated with the physiological data of the identified physiological sensor 18. When the physiological data of the physiological sensor 18 is collected by the monitor 20, it may be associated by the monitor 20 with the physiological sensor 18 in any suitable manner. As an example, a caregiver may manually input the identification data 16 to the monitor 20 via a keyboard, touch screen, writing pad, mouse, or the like. As another example, the identification data 16 may be stored or recorded in a bracelet (e.g., including a barcode, an RFID chip, a magnetic striped card, or the like) worn by the patient 14b. The monitor 20 may include a reader (e.g., a barcode reader, an RFID sensor, a card reader) configured to read out the identification data 16 from the bracelet and associated the identification data 16 with the sensor 18. The identification data 16 may be housed on a memory of the sensor 18 and may be transmitted to the monitor along with the physiological data. Further, the association may include bundling the received physiological data and the identification data 16 together, tagging the sensor data with a tag associated with the identification data 16, encrypting the sensor data with a key that is linked to the identification data, etc.

In the illustrated embodiment, a second physiological sensor, an unassociated sensor 12, gathers data from an unidentified patient 14a. The physiological data acquired by the unassociated physiological sensor 12 may include one or more physiological parameters or features in common with the physiological data of the associated sensor 18. For example, although the identified physiological sensor 18 and the unassociated physiological sensor 12 may be different types (e.g., one being an electrode-based heart rate sensor and the other being a pulse oximetry sensor), the same physiological parameters (e.g., heart rate) may be included in or extracted from the physiological data of both the identified physiological sensor 18 and the unassociated physiological sensor 12. In certain embodiments, the identified physiological sensor 18 and the unassociated physiological sensor 12 may be the same type of sensor.

The one or more common physiological parameters may be used to compare the physiological data of the identified physiological sensor 18 and the unassociated physiological sensor 12. If the one or more common physiological parameters are determined to be the same for both of the physiological data, the physiological data of the unassociated physiological sensor 12 is determined to be associated with the patient 14b. In this manner, the unidentified patient 14a is, once identified, indicated as an identified patient 14b. On the other hand, if the one or more common physiological parameters are determined to be different for both of the physiological data, the physiological data of the unassociated physiological sensor 12 is determined to not be associated with t the identified patient 14b. In other words, the unidentified patient 14a is determined to not be the patient 14b. If the unassociated physiological sensor 12 is determined to be associated with the patient 14b, the identification data 16 for identifying the patient 14a is then associated with the previously unassociated physiological sensor 12 and/or its corresponding physiological data. In other words, upon identification, the unassociated physiological sensor 12 is associated with the patient 14b. After the match is identified, linking the previously unassociated physiological sensor 12 to the associated or identified components of the system (i.e., patient 14a, sensor 18) may be performed in any suitable manner and according to any suitable data transmission protocol.

As disclosed herein, associating a sensor 12 with a patient 14 (and the patient's identification data 16) may include bundling the received physiological data and the identification data 16 together, co-communicating the sensor data and the patient identification data 16, tagging the sensor data with a tag associated with the identification data 16, encrypting the sensor data with a key that is linked to the identification data, etc. Further, in any of the disclosed embodiments, associating a sensor 12 with a patient 14 may include an association stored at the sensor 12, at the monitor 20, at a central monitoring station, in an electronic medical record, etc. The association may include storing identification information for the sensor 12 that is linked to the patient identification information 16. For example, such information may be stored in a look-up table or database. In one embodiment, the association may include writing patient identification data 16 to a memory of the sensor 12. In another embodiment, after the identification data 16 is written to the memory of the sensor 12, the measurement data from the physiological sensor 12 may be tagged or communicated together with patient identification data 16 for the patient 14b. Further, the monitor 20 may also store or display the identification data 16 along with calculated parameters from the sensor data. The association may also include providing the patient identification information 16 as part of a readable tag. For example, the readable tag may be a bar code or RFID tag that is provided as a label with patient records. In certain embodiments, when a sensor 12 is reused, a new association with a patient 14 overwrites any existing association.

It should be noted that “physiological sensor” as used herein refers to a medical device configured to detect one or more physiological parameters of a patient, such as patches, sensors, or probes. A medical monitoring system may also include a monitor (e.g., a medical monitor with a display, a processor, a memory, a power supply, an input device, and/or an alarm), and one or more connectors (e.g., power cable, data cable, and/or wireless communication devices such as a wireless transceiver) coupling the sensor and the monitor. The sensor, the monitor, and the one or more connectors may be separate from one another, or may be integrated into an integral unit (e.g., a portable oximeter, a portable heart rate monitor). As such, it should be noted that while each of the sensors 12 and 18 is illustrated in FIG. 1 as one unit coupled to the patient 14, each may include other components separate from the patient 14.

Physiological sensors as provided herein (e.g., sensor 12, sensor 18) may include optical, acoustic, electrical, or magnetic sensing components, or a combination thereof. The acquired physiological data may be transmitted to a monitoring component (e.g., monitor 20) that includes a processor and a memory configured to process the physiological data to generate a measure or indication of the one or more physiological parameters. As an illustrative example, the identified physiological sensor 18 is an electrocardiography (ECG) sensor. A sensing component includes one or more electrodes contacting the surface of the skin of the patient 14. In another example, the sensor 18 is a pulse oximetry sensor, and the sensing component includes a light emitter and photodetector.

The monitor 20, as discussed in greater detail below, may be a local monitor, a remote monitor, or may be a central monitoring station. Further, the monitor 20 may be a monitor worn by the patient. In such embodiments, the monitor 20 may receive signals and evaluate whether the received signals are associated with patient wearing the monitor 20. If not, the monitor 20 may then discard the unassociated signals. In contrast, the monitor 20 may be a monitor that collects data from multiple patients. In such embodiments, the received signals are kept and sorted between the various patients being monitored. The physiological sensors may be communicatively coupled to the monitor 20 in any suitable manner (e.g., via wired communication, wireless communication, or a combination thereof).

In certain embodiments of the disclosed techniques, at least one common feature or physiological parameter from physiological data between two or more sensors is compared to determine if the sensors are monitoring the same patient. The common feature may be an overlapping measured parameter (e.g., heart rate, blood pressure, respiration rate), a sequence of the overlapping measured parameter (e.g., a sequence of individual heart rate measurements over a period of time), a variability of the overlapping measured parameter (e.g., a ratio of a difference between a maximum value and a minimum value to the mean value of the overlapping parameter during a period of time), a trend (e.g., an increase, a decrease, or a pattern of fluctuation in amplitude or frequency) of the overlapping measured parameter, or any combination thereof.

In another embodiment of the disclosed techniques, associating sensors with a patient may be based on correlated physiological parameters from the physiological data of two or more sensors. The correlated physiological parameters described herein are physiological parameters, or waveforms or data associated with such parameters, that are not the same but respond in a correlated manner to one or more physiological events. As such, the correlated physiological parameters or their associated waveforms may change or vary (e.g., in amplitude, in frequency, in duration, in signal shape, or the like) in the same or similar manner during the same or similar time periods. These correlated physiological parameters can be used to associate a sensor with a patient. Further, the sequence, trend, or variability of such correlated parameters may also be used to identify or associate a sensor with a patient. Accordingly, one or more features or characteristics of the raw data from different physiological sensors may be compared to determine if they are associated with the same patient. For example, in an embodiment, the physiological data of the first physiological sensor (e.g., a heart rate sensor) includes an electrocardiography (ECG) waveform, and the physiological data of the second physiological sensor (e.g., a pulse oximetry sensor) includes a plethysmographic waveform. Although the ECG waveform is different from the plethysmographic waveform, both waveforms may include similar waveform features, such as decreasing peaks as a function of time, that respond to the same physiological event (e.g., an increased vascular resistance). A rhythm (e.g., frequency) of the occurrence of the repeating peaks may be compared between the ECG waveform and the plethysmographic waveform. In addition, one series of peaks may have a temporal relationship (e.g., a relatively fixed time delay) with respect to the other series of peaks. Accordingly, the physiological data of the second physiological sensor may be synchronized (e.g., time-shifted by the fixed time delay), and the correlated waveform features may be used to determine if the physiological of the second physiological sensor is associated with the first patient.

FIG. 2 illustrates one example of the types of data and/or parameters that may be compared between physiological data 22 from the identified physiological sensor 18 and the physiological data 24 from the second physiological sensor 12 of FIG. 1. As noted above, the first sensor, the identified physiological sensor 18, may be an ECG sensor, and the second sensor, an unassociated physiological sensor 12, may be a pulse oximetry sensor. The first set of physiological parameters 26 included in or determined from the physiological data 22 from the identified physiological sensor 18 may include heart rate 34a and ECG waveform 30 (e.g., including P waves, QRS complexes, T waves, and U waves). The second set of physiological parameters 32 included in or determined from the physiological data 24 from the unassociated physiological sensor 12 may include heart rate 34, plethysmographic waveform 36, and respiratory rate 38. As such, both the physiological data 22 from the identified physiological sensor 18 and the physiological data 24 from the unassociated physiological sensor 12 include a common physiological parameter, the heart rate 34a, 34b.

The heart rate 34a in the physiological data 22 from the identified physiological sensor 18 is associated with the patient 14. The heart rate 34b in the physiological data 24 from the unassociated physiological sensor 12 is then compared with the heart rate 34a to determine if the heart rate 34b is associated with the same patient 14. Comparison of common physiological parameter (e.g., the heart rate 34b with the heart rate 34a) may be based on any suitable characteristics of the common physiological parameter. For example, the characteristics of the heart rates 34a, 34b may include a value of the heart rate, a mean value over a time period, a trend value, a pattern or rhythm of the heart beats during a period of time, a variability of the heart rate (e.g., a ratio of a difference between a maximum value and a minimum value to the mean value of the heart rate during a period of time), or a combination thereof. In another embodiment, the common features may include the presence of common signal artifacts. For example, a patient in motion may cause motion artifacts in any sensor positioned on the patient. Therefore, the presence of a common signal artifact in unassociated physiological sensor 12 and the identified physiological sensor 18 may be indicative that they are associated with the same patient 14. In another embodiment, the presence of a common arrhythmia in unassociated physiological sensor 12 and the identified physiological sensor 18 may be indicative of an association with the same patient 14. In one embodiment, the identification of a common arrhythmia feature is based on a common arrhythmia type, such as a bradycardia or tachycardia as identified by sensors that measure pulse rate. For example, the presence of tachycardia as identified by an ECG and a plethysmographic sensor over a common time interval is indicative of a common arrhythmia. In one embodiment, the identification of a common arrhythmia feature is based on a presence or absence of common arrhythmia indicators in an ECG signal. In one embodiment, the identification of a common arrhythmia feature is based on a presence or absence of common arrhythmia indicators in a plethysmographic signal, such as those described in U.S. Pat. No. 8,755,871, the specification of which is incorporated by reference herein in its entirety for all purposes.

In certain embodiments, the comparison of the common physiological parameter is performed using data collected under the same or similar patient conditions. For example, the identified physiological sensor 18 and the unassociated physiological sensor 12 may operate at the same time window, when patients are in the same or similar positions, after potential associated patients are administered with the same medicine, or the like.

A tolerance level may be set for determining if the common physiological parameter is the same or different. For example, the tolerance level may be a threshold or a range. If the difference between the characteristics of the common physiological parameter from the physiological data 22 and from the physiological data 24 is within a pre-defined threshold or range, the characteristics of the common physiological parameter from both physiological data 22, 24 may be considered to be the same and associated with the same patient 14. On the other hand, if the difference between the characteristics of the common physiological parameter from the physiological data 22 and from the physiological data 24 is outside of the pre-defined threshold or range, the characteristics of the common physiological parameter from both physiological data 22, 24 may be considered to be different.

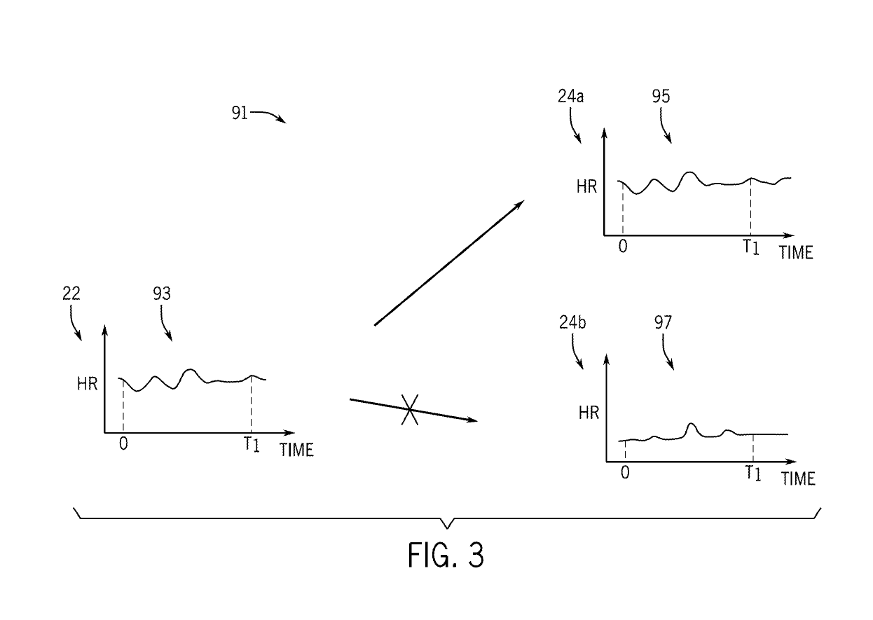

As noted above, the characteristics of the common physiological parameter (e.g., heart rate, blood pressure, respiration rate) may include a mean value, a variability, a sequence, a trend or a pattern, or any combination thereof, of the measured common physiological parameter from the physiological data 22 and from the physiological data 24. FIG. 3 illustrates a comparison 91 of a trend (or pattern) of a common physiological parameter (e.g., the heart rate) of the physiological data 22 from the identified sensor 18 and the physiological data 24a, 24b from the unassociated physiological sensor or sensors 12 to determine if the unassociated physiological sensor 12 is associated with the patient 14. As illustrated, the physiological data 22 includes a first chart 93 of the heart rate as a function of time. For example, the first chart 93 may include a series of heart rate values at various times during the time period of 0 to T₁. For example, the heart rate values may be obtained from the physiological data 22 (e.g., the ECG waveform 30) at fixed or variable time intervals (e.g., 0.5 s, 1 s, 2 s, 5 s, 10 s, 20 s, 30 s, 60 s, 90 s, 120 s, or the like).

Similarly, the trend of the heart rate may be obtained from the physiological data 24. A second chart 95 and a third chart 97 illustrate different embodiments of the heart rate as a function of time from the physiological data 24a, 24b (e.g., the plethysmographic waveform 36). The second chart 95 and the third chart 97 may include a series of heart rate values at various times during the time period of 0 to T₁. The heart rate values may be obtained from the physiological data 24a, 24b at fixed or variable time intervals that may be the same as or different from the time intervals used in the chart 93. As illustrated, the chart 95 of the physiological data 24a includes a trend (or pattern) of the heart rate during the time 0 to T₁ that is substantially the same (e.g., within a pre-determined tolerance level) as the trend (or pattern) of the heart rate illustrated in the chart 93. Accordingly, in the embodiment as illustrated in the chart 95, the trend or pattern of the heart rate may be used to determine that the data 24a of the unassociated physiological sensor 12 is associated with the patient 14. On the other hand, the chart 97 of the physiological data 24b includes a trend (or pattern) of the heart rate during the time 0 to T₁ that is different (e.g., outside a pre-determined tolerance level) from the trend (or pattern) of the heart rate illustrated in the chart 93. Accordingly, in the embodiment as illustrated in the chart 97, the trend or pattern of the heart rate may be used to determine that data 24b of the unassociated physiological sensor 12 is not associated with the patient 14. The determination may be made by an image comparison of the chart of heart rate values or by comparing various pattern features (e.g., peaks, valleys, etc.) of the values over time. For example, pattern features may be based on the presence of common metrics that may be derived from the sensor waveforms from a particular time period. Such metrics may be a comparison of values or variation between such values, including peak-to-peak values, peak-to-trough values, trough-to-trough values, area under the curve values, or pulse shape (e.g., skewness of the derivative of a plethysmographic signal).

It should be noted that the time window (e.g., the length of the time period T₁) for comparison of the trend (or pattern) of the common physiological parameter (e.g., the heart rate) may be predetermined or adjusted based on various factors, such as the characteristics of the common physiological parameter (e.g., frequency), characteristics of the sensors (e.g., sensitivity, accuracy), or data quality (e.g., collection time interval). It also should be noted that the trend (or pattern) of the common physiological data may be time-stamped, and for comparison, may be time-shifted with one another, as discussed in greater detail below. For example, in one embodiment, a heart rate from a pulse oximetry sensor is compared to a heart rate from an EKG. Although the calculation of the individual heart rate values in the pulse oximetry monitor and the EKG monitor may be time-shifted or at different time intervals (e.g., every 0.1 s vs. every 1 s) relative to one another, a chart of heart rate values over time may be used to extrapolate intervening heart rate values at a particular time point. Accordingly, in one embodiment, the matched pattern or sequence of heart rate values may be based at least in part on extrapolated heart rate values.

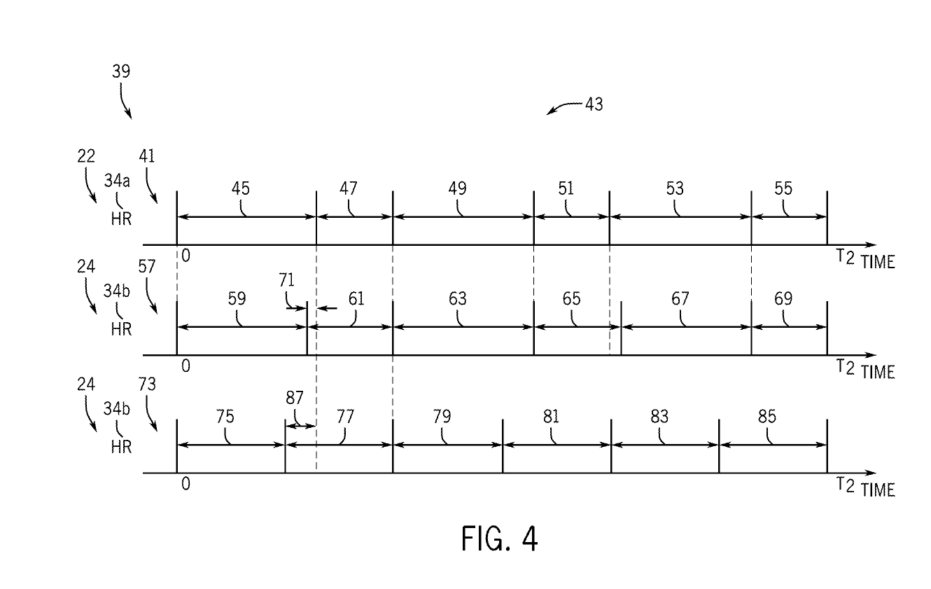

FIG. 4 illustrates another comparison 39 of a common physiological parameter (e.g., the heart rate) of the physiological data 22 from the identified sensor 18 and the physiological data 24 from the unassociated physiological sensor 12, based on the characteristics of the waveforms of the respective physiological data 22, 24, to determine if the unassociated physiological sensor 12 is associated with the patient 14. As illustrated, the heart rate 34a of physiological data 22 includes a sequence 41 of heart beats (simplified from the actual waveform and represented by vertical lines) during a time period 43 (e.g., from time 0 to time T₂). The sequence 41 includes a total number of 6 heart beats (e.g., not counting the heart beat at time 0) during the time period 43. The sequence 41 has a pattern of alternating long and short time separations between adjacent heart beats. For example, a relatively long time separation 45 is followed by a relatively short time separation 47, which is followed by alternating long time separations 49, 53 and short time separations 51, 55.

In one embodiment, the heart rate 34b of physiological data 24 may include a sequence 57 of heart beats during the time period 43. The sequence 57 also includes a total number of 6 heart beats (e.g., not counting the heart beat at time 0) during the time period 43. Accordingly, in this embodiment, the heart rate 34b may be determined to have the same mean value of heart rate as the heart rate 34a. In addition, the sequence 57 has a similar pattern of alternating long and short time separations between adjacent heart beats. For example, a relatively long time separation 59 is followed by a relatively short time separation 61, which is followed by alternating long time separations 63, 67 and short time separations 65, 69. Moreover, the time separations 59, 61, 63, 65, 67, 69 of the sequence 57 are substantially the same (e.g., within a predetermined a tolerance level) as the time separations 45, 47, 49, 51, 53, 55 of the sequence 41, respectively. For example, a difference 71 between the time separation 45 of the sequence 41 and the time separation 59 of the sequence 57 may be within the predetermined a tolerance level. Accordingly, the heart rate 34b may be determined to have the same pattern or trend as the heart rate 34a. As such, in this embodiment, the pattern or trend of the heart rate, in addition to the mean value of the heart rate, may be used to determine that the unassociated physiological sensor 12 is associated with the patient 14.

In another embodiment, the heart rate 34b of physiological data 24 may include a sequence 73 of heart beats during the time period 43. The sequence 73 also includes a total number of 6 heart beats (e.g., not counting the heart beat at time 0) during the time period 43. Accordingly, in this embodiment, the heart rate 34b may be determined to have the same mean value of heart rate as the heart rate 34a. However, the sequence 73 has a different pattern of the heart beats. For example, adjacent heart beats in the sequence 73 have substantially the same time separations (e.g., time separations 75, 77, 79, 81, 83, 85) with one another. Moreover, each time separation 75, 77, 79, 81, 83, 85 of the sequence 73 is different (e.g., outside of a predetermined tolerance level) from the corresponding time separation 45, 47, 49, 51, 53, 55 of the sequence 41. For example, a difference 87 between the time separation 45 of the sequence 41 and the time separation 75 of the sequence 73 may be outside of the predetermined a tolerance level. Accordingly, in this embodiment, although the heart rate 34b may be determined to have the same mean value as the heart rate 34a, the pattern or trend of the heart rate may be determined to be different. As such, based on the pattern or trend of the heart rate, the unassociated physiological sensor 12 may be determined not be associated with the patient 14.

Further, while the disclosed comparison techniques are discussed in the context of the heart rate, it should be understood that the comparison techniques may be applied to other physiological parameters to compare overlapping features. For example, a patient's blood pressure may be determined using one or more invasive and/or noninvasive techniques. In certain embodiments, a blood pressure as determined by an oscillometric technique from a first sensor may be compared to a blood pressure determined by a pulse wave velocity technique from a second sensor. In other embodiments, a patient's respiration rate as determined via a plethysmographic waveform from a first sensor may be compared to a respiration rate determined by capnography, transthoracic impedance, and/or impedance pneumography from a second sensor. Further, the compared parameters may be obtained using the results of multiple sensors. The physiological data 22 from the identified physiological sensor 18 and the physiological data 24 from the unassociated physiological sensor 12 may additionally or alternatively include one or more common physiological parameters of patients, such as heart rate, a variability of the heart rate, respiratory rate, a variability of the respiratory rate, ECG timing (e.g., timing of the QRS complex), blood pressure, response to medicine, or any combination thereof. As discussed in greater detail below, comparing the common physiological parameter from both physiological data 22, 24 to determine if the unassociated physiological sensor 12 is associated with the patient 14 may be performed by sensor (e.g., the sensor 12, 18) or by the monitor 20.

As discussed above, associating sensors with a patient may also be based on correlated physiological parameters from the physiological data of two or more sensors. The correlated physiological parameters are not overlapping or common physiological parameter, but respond in a correlated manner to one or more physiological events. As such, one physiological parameter may have a known and/or predictable impact on a second physiological parameter, and changes between the two physiological parameters may be correlated against each other to determine if they are associated with the same patient or not. As an example, if an increase of blood pressure (e.g., increased vascular resistance) of the patient 14 is detected in the ECG waveform 30 by the physiological sensor 18, and the physiological sensor 12 detects in the plethysmographic waveform 36 an increase in vascular resistance that is time correlated, then the physiological sensor 18 may be associated with the patient 14. In another example, the correlated parameter includes respiration rate derived from an ECG and the plethysmographic signal. Changes in respiration rate are known to have a specific impact on the plethysmograph, that may in certain embodiments be used to calculate the respiration rate from the plethysmograph. That is, in addition to or instead of using the calculated respiration rate parameter, the common feature may be the respiration-induced changes on a physiologic signal, with or without calculating the respiration rate from the pleth signal, the respiration rate-induced changes in the ECG and in the plethysmographic signal may be correlated to create an association or may be used as a correlated parameter. In other embodiments, the correlation may be an inverse correlation, depending on the feature used. For example, while certain physiological events may cause an increase in a measured parameter, the same event may cause a corresponding decrease in another parameter.

As noted above, the common physiological parameter (e.g., the heart rate 34a, 34 in FIG. 2) may be used for comparing the physiological data 22 from the identified physiological sensor 18 and the physiological data 24 from the unassociated physiological sensor 12. To perform the comparison, parameters measured at the same time point or within the same time window may be used for the comparison. That is, the compared data or measurements may be time-synchronized for the comparison by using time stamps associated with the data. Further, the compared data or measurements may be compared in real time, e.g., on a rolling basis, or based on retrospective data analysis. In some embodiments, one or more common characteristics or features of waveforms in the physiological data 22, 24 (e.g., waveform peaks or troughs) may also be directly compared (e.g., without first extracting the common physiological parameters from each of the physiological data 22, 24) to determine if the second physiological sensor 12 is associated with the patient 14. Because the physiological data 22, 24 may not be synchronized (e.g., respective waveform peaks or troughs not aligned with respect to time), the comparison of the physiological data 22, 24 may include synchronizing the physiological data 22, 24. For example, as illustrated in FIG. 2, the physiological data 22 includes the ECG waveform 30, and the physiological data 24 includes the plethysmographic waveform 36. The ECG waveform 30 and the plethysmographic waveform 36 may be synchronized and compared with one another against one or more common features or characteristics of the waveforms, such as a rhythm of repeating waveform peaks or troughs.

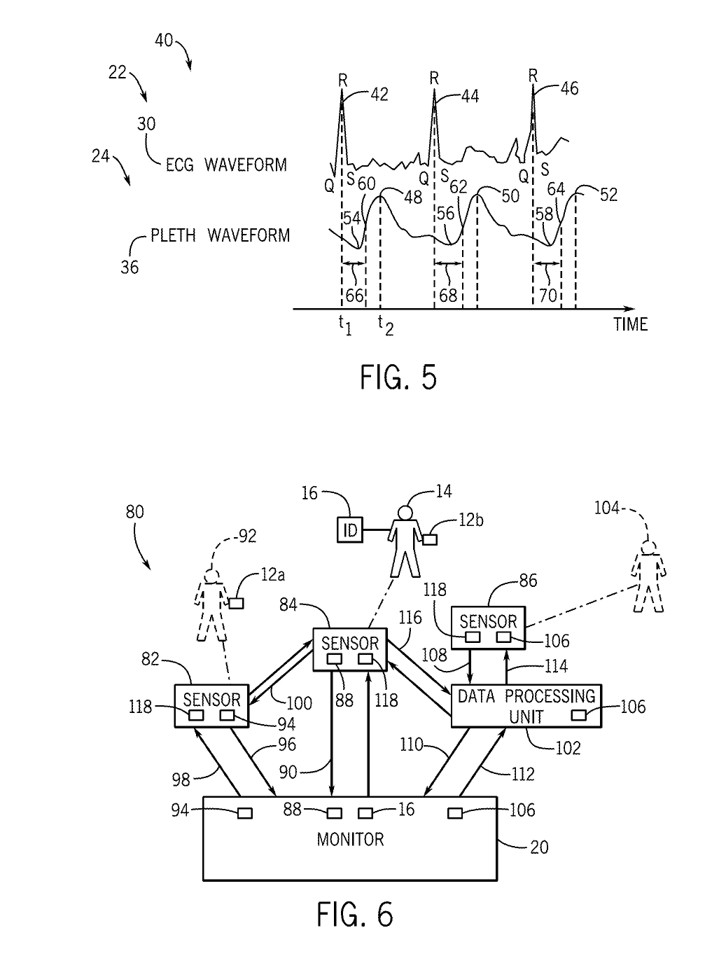

FIG. 5 illustrates a comparison 40 of physiological data 22 (e.g., the ECG waveform 30) from the identified sensor 18 and the physiological data 24 (e.g., the plethysmographic waveform 36) from the unassociated physiological sensor 12 to determine if the unassociated physiological sensor 12 is associated with the patient 14. As illustrated, the ECG waveform 30 includes the repeating QRS complexes (e.g., with peaks 42, 44, 46), representative of the depolarization of the right and left ventricles of the heart of the patient 14. The plethysmographic waveform 36 includes repeating peaks (e.g., peaks 48, 50, 52) and troughs (e.g., troughs 54, 56, 58) representative of the quantity of blood in an underlying blood vessel. As such, one common characteristic of both the ECG waveform 30 and the plethysmographic waveform 36 is repeating peaks, which may be used to compare the two waveforms 30, 36. From a physiological perspective, the ECG represents the electrical component of a “heartbeat” and in most instances results in nearly coincident mechanical contraction. The plethysmographic waveform is generally representative of the pressure waveform resulting from mechanical contraction, which is propagated through the vasculature and time shifted based on the speed of the pulse wave from the left ventricle to the point of measurement on the plethysmograph (e.g., the finger). Therefore the ECG (specifically the QRS complex) and the plethysmograph are time shifted, but correlated, events.

For an individual patient, there may be a variance in the interval between each heart beat (heart rate variation). This HRV creates a unique signature for each patient, which would be similar between the peaks of the plethysmographic waveform and the RR interval since both are driven by the same physiological process (electrical depolarization, as detected via an ECG, followed by pressure waveform, detected via a plethysmograph). The pulse wave velocity (effectively the time delay between QRS and plethysmograph peak) would be effectively constant as this is driven by the mechanical properties of the vasculature and in the absence of medication administration is unlikely to dramatically shift within a short period of time. Therefore, the pulse wave velocity may also be used as a common point of identification between two sensors, i.e., to associate an unassociated sensor with a sensor associated with a patient. Accordingly, a characteristic peak to peak distance (effectively pulse wave velocity or pulse transit time) between an ECG peak and a plethysmographic waveform peak may be a correlated feature. In addition, correlation of variability within the peaks may also be considered as maintenance of a relatively constant pulse wave velocity, or ECG peak to plethysmograph peak timing, within a relatively short duration.

In certain embodiments, the ECG waveform 30 and the plethysmographic waveform 36 may be feature-synchronized rather than time-synchronized to perform the comparison. For example, the first peak 42 of the ECG waveform 30 corresponds to a time t1, while the first peak 48 of the plethysmographic waveform 36 corresponds to a time t2. The waveforms may be unsynchronized because the physiological data 22, 24b are associated with different patients, or because even though the physiological data 22, 24a are associated with the same patient (e.g., the patient 14), there may be time delay or lagging between different types of physiological sensors. For example, if the plethysmographic wave form 36 is also associated with the same patient 14, there is a time delay (e.g., a pulse transit time) between the plethysmographic wave form 36 and the ECG waveform 30 due to transit time between a heartbeat and the induced blood pressure change at an extremity (e.g., finger) of the patient 14. For example, the plethysmographic wave form 36 includes multiple points 60, 62, 64 where the amplitude of the waveform crosses a threshold value (e.g., 20%, 25%, or 30%) of the amplitude from respective troughs 54, 56, 58 to peaks 48, 50, 52. The time differences 66, 68, 70 between the peaks 42, 44, 46 of the ECG waveform 30 and the corresponding points 60, 62, 64 of the plethysmographic waveform represent pulse transit times. Accordingly, comparing the common characteristics of both the ECG waveform 30 and the plethysmographic waveform 36 (e.g., their repeating peaks) may include feature-synchronizing the ECG waveform 30 and the plethysmographic waveform 36, for example, by time-shifting one of the waveforms 30, 36 with respect to another (e.g., by a characteristic time difference, such as difference 66, representative of the physiological delay). If the waveforms 30, 36 align when time-shifted by the characteristic time difference 66, then they may be more likely to be from the same patient 14. If they align only when shifted more than the characteristic time difference 66, then they may be less likely to be from the same patient 14. In some embodiments, synchronization of the physiological data 22, 24 may include time-stamping both of physiological data 22, 24, and comparing the physiological data 22, 24 may include comparing the one or more characteristics of features of the physiological data 22, 24 at the same time or during the same time window based on the time stamps.

Any suitable characteristics or features of the physiological data 22, 26 may be used for synchronization and comparison of the physiological data 22, 26, including a repeating rhythm of the peaks (or troughs, or any other graphic indicators), a pattern or trend of the peaks (or troughs, or any other graphic indicators), a presence, absence, appearance, or disappearance of peaks (or troughs, or any other graphic indicators) induced by an external stimulus (e.g., application of a pressure cuff to cause temporary disappearance of a plethysmographic signal), or any combination above. It also should be noted that the ECG waveform 30 and the plethysmographic waveform 36 are used as non-limiting examples, and the physiological data 22, 24 used for synchronization and comparison may include any suitable type of physiological data from various type of physiological sensors.

Similarly as described with respect to FIG. 2, a tolerance level (e.g., a threshold or a range) may be set for determining if the one or more common characteristics or features are the same or different for the physiological data 22, 24. If the one or more common characteristics or features for both physiological data 22, 24 are determined to be the same, the physiological data 24 from the second physiological sensor 12 is determined to be associated with the same patient 14. In other words, the second physiological sensor 12 is determined to be associated with the same patient 14. If the one or more common characteristics or features for both physiological data 22, 24 are determined to be different, the physiological data 24 from the second physiological sensor 12 is determined to not be associated with the patient 14. The synchronization and comparison of the physiological data 22, 24 may be performed by the second physiological sensor 12 or by the monitor 20.

FIG. 6 illustrates an embodiment of a medical monitoring system 80 for associating physiological sensors (e.g., the second physiological sensor 12 of FIG. 1) with patients (e.g., the patient 14 of FIG. 1). The medical monitoring system 80 includes the monitor 20 and multiple physiological sensors. While the multiple physiological sensors are illustrated that include first and second sensors 12a, 12b and a third physiological sensor 82, a fourth physiological sensor 84, and a fifth physiological sensor 86, it should be noted that the medical monitoring system 80 may include any number of physiological sensors. It also should be noted that the multiple physiological sensors may be coupled to the same patient or different patients.

As illustrated, the fourth physiological sensor 84 is coupled to the patient 14 to detect and monitor one or more physiological parameters of the patient 14. Accordingly, the identification data 16 of the patient 14 may be associated with physiological data 88 acquired by the fourth physiological sensor 84. The fourth physiological sensor 84 may store and the physiological data 88 and the associated identification data 16 and/or transmit both data 88, 16 to the monitor 20 (e.g., as indicated by an arrow 90).

The third physiological sensor 82 is coupled to an unidentified patient 92, who may be the patient 14 or a different patient. The third physiological sensor 82 acquires physiological data 94 of the unidentified patient 92 and may store the physiological data 94 and/or transmit the physiological data 94 to the monitor 20 (e.g., as indicated by an arrow 96). In accordance with the techniques described above with respect to FIGS. 1-5, the physiological data 94 may be compared to the physiological data 88 to determine if the physiological data 94 from the third physiological sensor 82 is associated with the patient 14, in other words, if the unidentified patient 92 is the patient 14 or a different patient. More specifically, as discussed above, for determining the association of the third physiological sensor 82 with the patient 14, one or more common or correlated physiological parameters of the physiological data 88 and 94 may be compared. Additionally or in the alternative, the physiological data 88 and 94 may be synchronized and one or more common or correlated characteristics or features of the physiological data 88 and 94 may be compared.

In some embodiments, the monitor 20, after acquiring the identification data 16 of the patient 14, the physiological data 88 from the fourth physiological sensor 84, and the physiological data 94 from the third physiological sensor 82, compares the physiological data 88 and 94 to determine if the third physiological sensor 82 is associated with the patient 14. If the monitor 20 determines that the third physiological sensor 82 is associated with the patient 14, the monitor 20 may assign the third physiological sensor 82 to the patient 14 by transmitting the identification data 16 of the patient 14 to the third physiological sensor 82 (e.g., as indicated by an arrow) or by assigning the identification data 16 to the third sensor 82 internally in the monitor 20. In other embodiments, the third physiological sensor 82 fetches the physiological data 88 and the identification data 16 of the patient 14 from the fourth physiological sensor 84 (e.g., as indicated by an arrow 100) and compares the physiological data 88 and 94 at the third physiological sensor 82 to determine if the third physiological sensor 82 is associated with the patient 14. If the third physiological sensor 82 determines that the third physiological sensor 82 is associated with the patient 14, the third physiological sensor 82 may assign itself to the patient 14 by associating the physiological data 94 with the identification data 16 of the patient 14 and/or transmitting both the physiological data 94 and the identification data 16 to the monitor 20.

The medical monitoring system 80 may also include one or more data processing units 102 coupled to one or more physiological sensors. For example, a data processing unit 102 couples the fifth physiological sensor 86 and the monitor 20. The data processing unit 102 is configured to process (e.g., analog-to-digital convert, digital-to-analog convert, filter, amplify, or the like) physiological data acquired by the fifth physiological sensor 86 and communicate the raw and/or the processed physiological data between the fifth physiological sensor 86 and the monitor 20. The data processing unit 102 may be a standalone unit (e.g., a computer, or another monitor), or may be integrated with either the fifth physiological sensor 86 or the monitor 20. The data processing unit 102 may include hardware components such as a processor, an amplifier, a receiver, a transmitter, a cable, etc. As another example, the fifth physiological sensor 86 and the data processing unit 102 are located in a patient room, and the monitor 20 is located in a remote control room. In such embodiments, the monitor 20 may be implemented as a central monitoring station.

The fifth physiological sensor 86 is coupled to an unidentified patient 104, who may be the patient 14 or a different patient. The fifth physiological sensor 86 acquires physiological data 106 of the unidentified patient 104 and may store the physiological data 106 and/or transmit the physiological data 106 to the data processing unit 102 and the monitor 20 (e.g., as indicated by arrows 108, 110). Similarly, the physiological data 106 may be compared to the physiological data 88 to determine if the physiological data 106 from the fifth physiological sensor 86 is associated with the patient 14, in other words, if the unidentified patient 104 is the patient 14 or a different patient. In some embodiments, the monitor 20 compares the physiological data 88 and 106 to determine if the fifth physiological sensor 86 is associated with the patient 14. If the monitor 20 determines that the fifth physiological sensor 86 is associated with the patient 14, the monitor 20 may assign fifth physiological sensor 86 to the patient 14 by transmitting the identification data 16 of the patient 14 to the fifth physiological sensor 86 (e.g., as indicated by arrows 112, 114). In other embodiments, the data processing unit 102 fetches the physiological data 88 and the identification data 16 of the patient 14 from the fourth physiological sensor 84 (e.g., as indicated by an arrow 116) and compares the physiological data 88 and 106 at the data processing unit 102 to determine if the fifth physiological sensor 86 is associated with the patient 14. If the data processing unit 102 determines that the fifth physiological sensor 86 is associated with the patient 14, the data processing unit 102 may assign the fifth physiological sensor 86 to the patient 14 by associating the physiological data 106 with the identification data 16 of the patient 14 and/or transmitting both the physiological data 106 and the identification data 16 to the monitor 20.

Each of multiple physiological sensors in the medical monitoring system 80 may include an indicator 118 (e.g., a display, a light, an alarm, or a combination thereof) configured to indicate (e.g., with a certain image, pattern, sound, or color) if the respective physiological sensor is associated with an identified patient. Also, it should be noted that all of the communication (e.g., data transfer) in the medical monitoring system 80 (e.g., as indicated by the arrows 90, 96, 98, 100, 108, 110, 112, 114, 116) may include any suitable manner, such as wired communication, wireless communication, or a combination thereof.

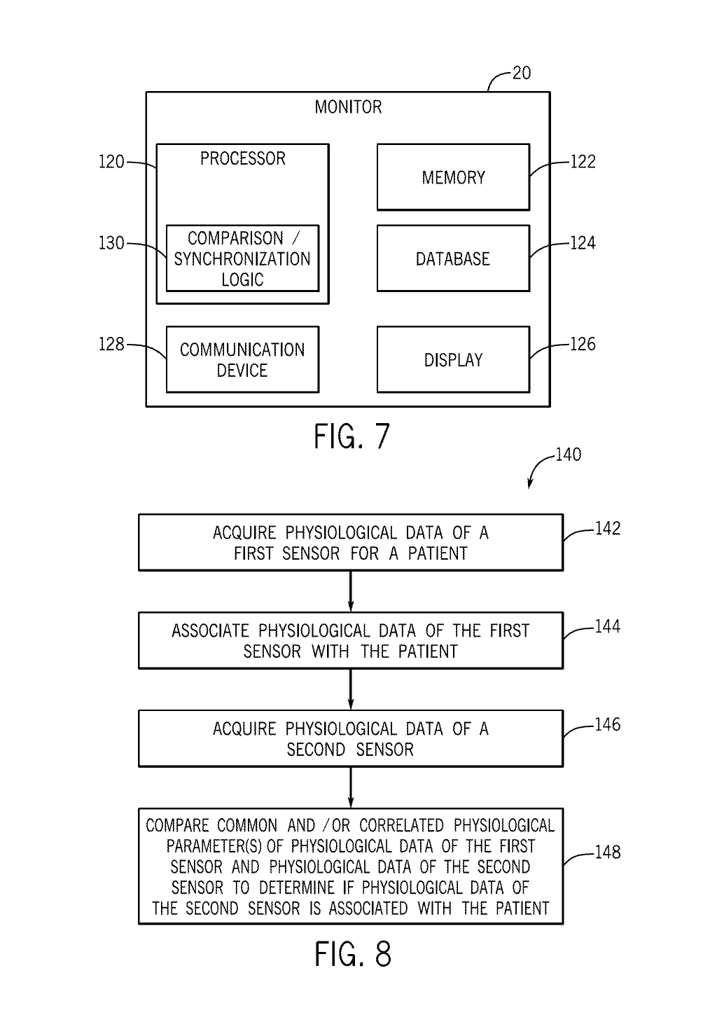

FIG. 7 illustrates an embodiment of the monitor 20 implemented as a central monitoring station and including certain hardware features such as a processor 120, a memory 122, a database 124, a display 126, and a communication device 128 to facilitate associating one or more physiological sensors to one or more patients. It should be understood that other implementations of the monitor 20, e.g., as a bedside monitor, a standalone monitor, etc., may also include similar features. The memory 122 may include one or more tangible, non-transitory, machine-readable media collectively storing instructions executable by the processor 120 to perform the methods and techniques described herein. Such machine-readable media may include any suitable media, such as RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by the processor 120 or by any general purpose or special purpose computer or other machine with a processor. The memory 122 may also store physiological data acquired by one or more physiological sensors (e.g., the identified physiological sensor 18, the second physiological sensor 12, the third physiological sensor 82, the fourth physiological sensor 84, and the fifth physiological sensor 86) in the medical monitoring system 80. Moreover, the memory 122 may store identification data for patients, such as the identification data 16 of the patient 14.

The processor 120 may be a general purpose or an application-specific processor and may include one or more processing devices. The processor 120 may be configured to process the physiological data acquired by the one or more physiological sensors and the identification data for the patients to associate the one or more physiological sensor to the patients in accordance with the techniques described herein. The processor 120 may include comparison and/or synchronization logic 130 configured to perform comparisons and/or synchronize the physiological data from multiple physiological sensors. The comparison/synchronization logic 130 may execute any suitable instructions or algorithms (e.g., stored in the memory 122). For synchronization, the physiological data may be properly time-stamped when being collected by the respective physiological sensors or when being transmitted to the monitor 20. The processor 120 (e.g., the comparison/synchronization logic 130) may then compare the one or more common characteristics or features of the physiological data to determine if the unidentified physiological sensor is associated with a patient (e.g., the patient 14).

The monitor 20 may also include a database 124 (e.g., an electronic medical record) storing identification data of unique patients (e.g., name, age, gender, date of birth, and admission date) and the medical record, including previously collected physiological data from one or more physiological sensors associated with the patients. Accordingly, unidentified (e.g., associated with an unidentified patient and not with a unique patient) physiological data may be acquired by the central monitoring station and be compared with the medical record stored in the database 124 to determine if the unidentified physiological data is associated with any particular patient in the system (e.g., with identification data for the particular patient stored in the database 124). The database 124 may be separate from or integrated with the memory 122.

The display 126 of the central monitoring station may be configured to display any information on the physiological data from the physiological sensors in the medical monitoring system 80, including the physiological parameters, characteristics, or features of the physiological data. The display 126 may also display information on the patients, including the identification data, together with one or more displayed parameters. For example, the display 126 may display parameter data and patient identification information for a sensor identified to be associated with the identification information (and, in turn, the patient). When a new sensor is associated with the identification information, the parameter data from the newly associated sensor may, upon association, then be displayed together with the previously-displayed information. Accordingly, a display screen with n displayed parameters may automatically update to display n+1 parameters when a new sensor is associated with the patient whose parameters are being displayed. The display 126 may further include an indicator (e.g., a graph, an alarm, or a combination thereof) configured to indicate (e.g., with a certain pattern, or sound) if particular physiological data or sensor is associated with an identified patient. The communication device 128 of monitor 20 may include any suitable device for communicating (e.g., data transfer) the monitor 20 to the physiological sensors or data processing units via wired communication, wireless communication, or a combination thereof. For example, the communication device 128 includes a cable, a cable port, a wireless transceiver, a Bluetooth device, or the like.

FIG. 8 is a flow diagram of a method 140 for associating a physiological sensor (e.g., the unassociated physiological sensor 12, the third physiological sensor 82, and the fifth physiological sensor 86) to a patient (e.g., the patient 14) in accordance with embodiments of the present disclosure. The method 140 may start with acquiring physiological data of a first sensor (block 142). The first sensor may be coupled to the patient 14 to detect and monitor one or more physiological parameters of the patient 14. The physiological data (e.g., the physiological data 22, 88) of the first sensor may then be associated with the patient 14 (block 144), e.g., by transmitting the identification data 16 to a central monitoring station to be associated with the physiological data of the first sensor. A second sensor (e.g., the unassociated physiological sensor 12, the third physiological sensor 82, or the fifth physiological sensor 86) may be coupled to an unidentified patient to acquire physiological data for detecting and monitoring one or more physiological parameters of the unidentified patient (block 146). The unidentified patient may be the same patient 14 or a different patient. In certain embodiments, the method 140 acquires data from any sensors in a defined wireless transmission range or that are within a network of the monitor 20. Alternatively or additionally, the monitor 20 may collect or fetch data based on wireless signal strength of the unassociated sensor relative to a device associated with a candidate patient. For example, in one embodiment, the collected data may be ranked according to wireless signal strength relative to a sensor associated with a candidate patient or a monitor local to the patient. Potential matches that are determined to be closer to the candidate patient may be assessed first. Alternatively, signal strength may be used as a validation for association. If a sensor is matched with a patient, but the wireless signal strength relative to a local device is indicative of poor matching, an alarm or error message may be triggered.

When the physiological data of the first sensor and the second sensor include one or more common physiological parameters (e.g., heart rate, variability of the heart rate, respiratory rate, and variability of the respiratory rate), the one or more common physiological parameters may be used to compare the physiological data of the first sensor and the second sensor to determine if the physiological data of the second sensor is associated with the patient 14 (block 148). In certain embodiments, in addition to or alternatively, one or more correlated physiological parameters may be compared to determine if the physiological data of the second sensor is associated with the patient 14. As discussed above, comparison of the common physiological parameter may be based on any suitable characteristics (e.g., a value, a pattern, or a trend) of the common physiological parameter. A tolerance level (e.g., a threshold or a range) may be set for determining if the common physiological parameter is the same or different. If the characteristics of the common physiological parameter from the physiological data of the first sensor and the second sensor are determined to be the same, the physiological data of the second sensor, and consequently the second sensor itself, is determined to be associated with the patient 14. On the other hand, if the characteristics of the common physiological parameter from the physiological data of the first sensor and the second sensor are determined to be different, the physiological data of the second sensor, and consequently the second sensor, is determined to not be associated with the patient 14. In certain embodiments, the method 140 may include a validation step. For example, if the unassociated sensor is matched to a patient already associated with a sensor of the same type (e.g., if the unassociated sensor and an associated sensor are both pulse oximetry sensors), the method 140 may generate a flag for a manual check. As another example, if the unassociated sensor is matched to a patient already associated with a first sensor that has a much weaker wireless signal (possibly indicating that the first sensor is a larger distance away), the method may generate a flag for a manual check.

In certain embodiments, the physiological data of the first sensor and the second sensor may be synchronized prior to or in conjunction with the comparison. For example, the synchronization may include time-shifting or feature-shifting the physiological data of the first sensor and the second sensor relative to one another. After synchronization, the physiological data of the first sensor and the second sensor may be compared with respect to the one or more common characteristics or features (e.g., a repeating rhythm of peaks, a pattern or trend of peaks, a presence, absence, appearance, or disappearance of peaks) to determine if the physiological data of the second sensor is associated with the patient 14.

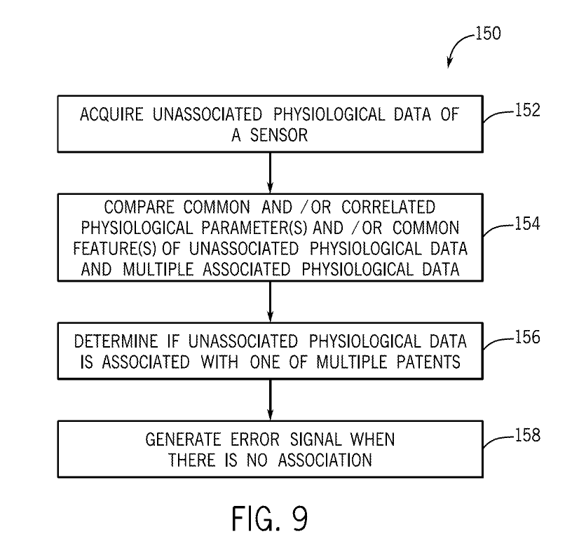

In addition to direct comparison between two sensors, the present techniques may be used to associate a sensor with a particular patient among a pool of candidate patients. FIG. 9 is a flow diagram of a method 150 for associating a physiological sensor (e.g., the second physiological sensor 12, the third physiological sensor 82, and the fifth physiological sensor 86) to a patient (e.g., the patient 14) in accordance with embodiments of the present disclosure. The method 150 acquires the physiological data of an unassociated sensor (block 152) and compares one or more common features or parameters of the unassociated physiological data to a pool of candidate physiological data (block 154) to determine if there is a match (block 156). If there is no match, the method 150 may provide an error signal (block 158) or other indication to the caregiver. In certain embodiments, in addition to or alternatively, one or more correlated physiological parameters may be compared to determine if there is a match.

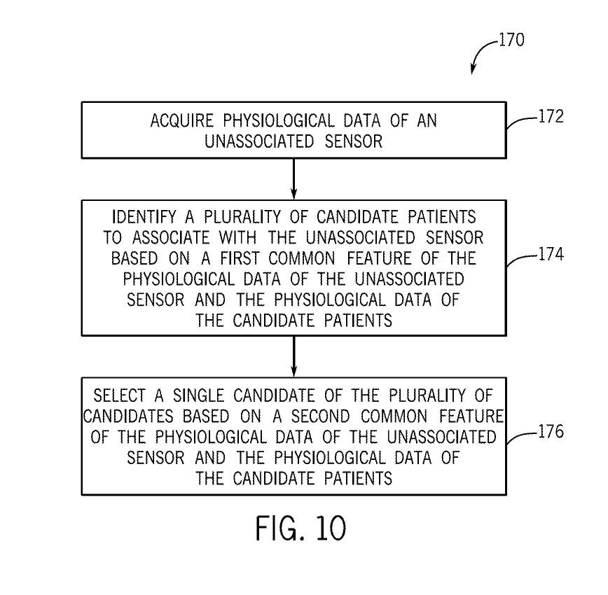

In certain cases, depending on the common parameter used for association, an unassociated sensor may match with multiple patients in a pool of candidates. FIG. 10 is a flow diagram of a method 170 in which unassociated sensor data is acquired (block 172) and a common feature of the unassociated data (e.g., a measured parameter or a characteristic of the data signal) as compared collected sensor data from a group of patients is used to identify a patient in the group of patients with which to associate the sensor. When the common feature identifies more than one potential match among the group of patients (block 174), the method 170 progresses to narrow the pool to a single match by evaluating a second common feature among the group from the initial match and selects a single candidate based on the second common feature (block 176). For example, a group of patient in an ICU may include several patients that have relatively matched heart rates. Accordingly, the method 170 may also use heart rate variability to narrow the group to a single match. In one embodiment, the second common feature is a derivate of the first common feature (i.e., heart rate and heart rate variability). In certain embodiments, in addition to or alternatively, identification and association may be based on one or more correlated physiological parameters (e.g., in block 174, or block 176, or a combination thereof).

The disclosed techniques may be used validate manual entry of patient identification data. In one example, a caregiver applies a sensor to a patient and manually inputs the association, e.g., at a central station, bedside monitor, or at the sensor itself). A manually-associated sensor may be validated against other sensors associated with the same patient. For example, all sensors associated with a single patient may undergo a validation check in which common features and/or parameters are used to determine if the sensors pass a “same patient” check. The check may be performed at the start of monitoring or when a new sensor is detected by the system (e.g., system 10, see FIG. 1) as being associated with a particular patient. If the sensor data as compared to the other sensors on the patient is consistent with all of the sensors monitoring the same patient based on the disclosed techniques, an approval message may be generated. In the alternative, if the newly-associated sensor does not appear to be measuring the same patient as the other sensors, an error message is generated, which may in turn trigger an alarm. The alarm may alert the caregiver that either the newly-associated sensor or a previously-associated sensor was improperly associated with a particular patient.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims. Further, it should be understood that elements of the disclosed embodiments may be combined or exchanged with one another.

What is claimed is:

1. A method for assigning a physiologic sensor to a patient, comprising:
monitoring a patient with a plethysmograph sensor detecting a plethysmograph signal from the patient;
monitoring the patient with an electrode sensor detecting an electrical signal from the patient;
receiving both the plethysmograph signal and the electrical signal at a remote monitor;
detecting, at the remote monitor, a sequence of heart beats in both the plethysmograph signal and the electrical signal;
matching, at the remote monitor, a sequence of the detected heart beats in the plethysmograph signal with a sequence of the detected heart beats in the electrical signal; and
assigning, at the remote monitor, both sensors to the same patient based on the matching features.

2. The method of claim 1, wherein matching the sequences comprises detecting a similar pattern of heart beats in the plethysmograph and the electrical signals, within a set tolerance level.

3. The method of claim 1, wherein assigning both sensors to the same patient comprises storing a patient identifier with physiologic data from the plethysmograph sensor and storing the same patient identifier with physiologic data from the electrode sensor.

4. The method of claim 3, wherein storing the patient identifier with physiologic data from the plethysmograph sensor comprises writing the patient identifier to a memory of the plethysmograph sensor.

5. The method of claim 3, wherein storing the patient identifier with physiologic data from the electrode sensor comprises writing the patient identifier to a memory of the electrode sensor.

6. The method of claim 3, wherein storing the patient identifier with physiologic data from the plethysmograph sensor and the electrode sensor comprises writing the patient identifier to a memory of the remote monitor in a database or lookup table and associated with identification information for the plethysmograph sensor and the electrode sensor.

7. The method of claim 1, wherein assigning both sensors to the same patient comprises accessing a patient identifier associated with the plethysmograph sensor or the electrode sensor and storing the patient identifier with physiologic data from the electrode sensor and the physiologic data from the plethysmograph sensor.

8. The method of claim 1, wherein the plethysmograph sensor and the electrode sensor are both wireless sensors.

9. The method of claim 8, further comprising wirelessly transmitting the plethysmograph signal from the plethysmograph sensor to the remote monitor.

10. The method of claim 1, comprising displaying the patient identifier together with a first heart rate determined from the detected heart beats from the electrical signal and a second heart rate from the detected heart beats of the plethysmograph signal.

11. The method of claim 1, wherein the matching comprises determining a first heart rate from the sequence of the detected heart beats in the plethysmograph signal and a second heart rate of the sequence of the detected heart beats in the electrical signal and matching the first heart rate and the second heart rate within a set tolerance level.

12. The method of claim 1, wherein the matching comprises determining a first heart rate variability from the sequence of the detected heart beats in the plethysmograph signal and a second heart rate variability of the sequence of the detected heart beats in the electrical signal and matching the first heart rate variability and the second heart rate variability within a set tolerance level.

13. A method for identifying a new wireless sensor, comprising:
receiving, at a patient monitor, physiologic data from a plurality of sensors each respectively monitoring a unique patient;
receiving, at the patient monitor, new physiologic data from a new sensor not yet assigned to a patient;
extracting a portion of the new physiologic data over a time duration and searching for a matching portion in the physiologic data from the plurality of sensors over the same time duration;
identifying a match with a first sensor of the plurality of sensors;
assigning the new sensor to the unique patient monitored by the first sensor; and
displaying incoming physiologic data from the new sensor together with incoming physiologic data from the first sensor.

14. The method of claim 13, comprising displaying identification information for the unique patient with the incoming physiologic data from the new sensor and the incoming physiologic data from the first sensor.

15. The method of claim 13, wherein identifying a match comprises determining a physiological parameter, a correlated physiological parameter, a variability of the common physiological or the correlated physiological parameter, a trend of the common physiological or the correlated physiological parameter, a common or correlated waveform feature, a pattern of the common or correlated waveform feature, or any combination thereof for the new sensor and the first sensor.

16. The method of claim 13, wherein identifying a match comprises matching a common parameter value derived from the physiologic data of the new sensor over the time duration to matching parameter value of in the physiologic data from the first sensor over the same time duration.

17. A system, comprising:
wireless communication circuitry configured to receive a first input signal from a first physiological sensor and a second input signal from a second physiological sensor;
a memory storing identification data for a patient and association data associating the first physiological sensor and the second physiological sensor with the identification data; and
a processor configured to:
receive the first input signal from the first physiological sensor associated with the patient;
compare a common feature in the first input signal from the first physiological sensor and in the second input signal from the second physiological sensor to determine if the second physiological sensor should be associated with the patient; and
generate an error message when the first physiological signal and the second physiological signal should not be associated with the patient based on the common feature.

18. The system of claim 17, wherein the first physiological sensor is a different sensor type from the second physiological sensor.

19. The system of claim 17, wherein the common feature is a common physiological parameter, a correlated physiological parameter, a variability of the common physiological or the correlated physiological parameter, a trend of the common physiological or the correlated physiological parameter, a common or correlated waveform feature, a pattern of the common or correlated waveform feature, or any combination thereof.

20. The system of claim 19, wherein the processor is configured to synchronize the first input signal with the second input signal before determining the common feature.
